(19) United States
(12) Patent Application Publication
(10) Pub. No.: US 2026/0355061 A1
(43) Pub. Date: Nov. 12, 2026

(54) BREAST MILK MANAGEMENT SYSTEM AND USES THEREOF

(71) Applicant: Nooshee Inc., Raleigh, NC (US)

(72) Inventors: Laura Haft, Raleigh, NC (US); William C. Brody, Raleigh, NC (US); Heath Stephenson, London (GB)

(73) Assignee: Nooshee Inc., Raleigh, NC (US)

(21) Appl. No.: 19/515,095

(22) PCT Filed: Aug. 30, 2024
(86) PCT No.: PCT/US2024/044911

§ 371 Date: Feb. 27, 2026

Related U.S. Application Data
(60) Provisional application No. 63/535,915, filed on Aug. 31, 2023.

(51) Int. Cl. A61J 9/00; G01N 33/487

(52) U.S. Cl. CPC A61J 9/00; G01N 33/48771

(57) ABSTRACT

A breast milk management system includes a storage unit for storing breast milk, the storage unit including a storage unit tag. The breast milk management system further includes at least one processing circuit comprising at least one processor and at least one memory having instructions thereon that, when executed by the at least one processor, cause the at least one processor to receive information associated with the breast milk based on the storage unit tag being scanned by a user device of a user. The instructions, when executed by the at least one processor, further cause the at least one processor to determine a characteristic of the breast milk based on the information associated with the breast milk. The instructions, when executed by the at least one processor, further cause the at least one processor to provide an indication of the characteristic to the user via the user device.

### CROSS-REFERENCE TO RELATED APPLICATION

This application claims the benefit of and priority to U.S. Provisional Patent Application No. 63/535,915, filed on Aug. 31, 2023, which is incorporated herein by reference in its entirety.

### BACKGROUND

Breastmilk is uniquely advantageous to infants, from providing passive immunity in the form of antibodies, potentially lower risk of sudden infant death syndrome (SIDS), and gastrointestinal infections, to potentially reduced risks of asthma and obesity later in life.

Many new mothers use breast pumps post-birth in order to achieve their personal milk feeding and expression goals. Breast pumps systems draw breast milk from a breast of a user and may be used to pump breast milk that may be stored for later consumption by an infant. Authorities including the Centers for Disease Control, and the American Academy of Pediatrics release guidelines for safe milk storage at a variety of temperatures. Pumping and storing breast milk for later use requires a significant time commitment and level of organization to ensure that milk is properly stored and used within the time frames set by these guidelines. The consequences for mislabeling a storage unit of breast milk range from erroneously disposing of a safe and nutritious aliquot of breast milk, to feeding expired or possibly contaminated breastmilk to a child.

### SUMMARY

A current practice for tracking of stored breast milk is to label the milk storage units individually by hand, and then enter information regarding the date, time, storage unit, and storage conditions into a personal log that will track only the input information. Whilst this is an effective method to store the milk, currently any attempt to log and track each bag of milk is purely manual and prone to human error, resulting in milk having to be disposed of due to safety issues. There are no commercially available systems that automate any parts of the milk storage pipeline. Thus, there is a need for breast milk management systems that can automate and streamline milk tracking to ensure timely milk use and safe milk storage.

The present disclosure provides a breast milk management system, components thereof, and uses thereof that allow for various information to be automatically logged and tracked and provide a variety of benefits over traditional break milk management strategies.

One embodiment relates to a breast milk management system. The breast milk management system includes a storage unit for storing breast milk, the storage unit including a storage unit tag. The breast milk management system further includes at least one processing circuit comprising at least one processor and at least one memory having instructions thereon that, when executed by the at least one processor, cause the at least one processor to receive information associated with the breast milk based on the storage unit tag being scanned by a user device of a user. The instructions, when executed by the at least one processor, further cause the at least one processor to determine a characteristic of the breast milk based on the information associated with the breast milk. The instructions, when executed by the at least one processor, further cause the at least one processor to provide an indication of the characteristic to the user via the user device.

Another embodiment relates to a method of managing breast milk. The method includes receiving information associated with breast milk stored within a storage unit having a storage unit tag based on the storage unit tag being scanned by a user device of a user. The method further includes determining a characteristic of the breast milk based on the information associated with the breast milk. The method further includes providing an indication of the characteristic to the user device.

In one aspect, the disclosure provides a breast milk management system including a) a storage unit for storing breast milk and including a storage unit tag; and b) a physical computing device including one or more processors in communication with a computer readable memory being programmed with executable instructions. The executable instructions may be programmed to (i) receive information from the storage unit tag; (ii) store information from the storage unit tag; and (iii) determine the length of storage of the storage unit, e.g., an expiration date.

In some embodiments, the breast milk management system may include a plurality of storage units and a plurality of storage unit tags for storing breast milk, wherein each storage unit corresponds to one storage unit tag. Information from the plurality of storage unit tags may be stored and organized in a database. In some embodiments, information about the storage unit tag stored and organized in the database may include an expiration date for the milk stored in a storage unit, time, date, and/or location of breast pumping and/or time, and/or location of storage of the storage unit, and/or temperature. Inventory of the storage units included in the database may be displayed on the physical computing device, e.g., visually. In some embodiments, the database may be configured such that a user may search or reorganize the database and/or information displayed based on personal preference.

In some embodiments, the executable instructions may be further programmed to calculate an expiration date for milk stored in a storage unit. In some embodiments, the executable instructions may be further programmed to identify one or more storage units for use or disposal. For example, the executable instructions may identify the oldest unexpired milk for consumption, e.g., first in first out. In another example, the executable instructions may be programmed to account for the time of day, prioritizing breast milk pumped at night for a nighttime feeding.

In some embodiments, the executable instructions may be programmed to produce an alert, e.g., an alarm. The alert may be sent to the physical computing device and/or the storage unit tag. In some embodiments, the physical computing device and/or the storage unit tag may display an alert after receiving a signal.

In some embodiments, the storage unit tag may include one or more of a 1D barcode label, a 2D barcode label, a radio frequency label (RFID), a beacon-based label, a near field communication (NFC) tag, a Bluetooth label, a magnetic label, an optical character recognition label, a digital label such as a QR code, a character-based label, a color label, or a symbol label. The storage unit tag may include one or more of a computer-readable signal and/or a human-readable signal. In some embodiments, the storage unit tag may include a computer-readable memory.

In some embodiments, the storage unit may include a temperature sensor. In some embodiments, the temperature sensor may be disposed in the storage unit. In some embodiments, the temperature sensor may be disposed in the storage unit tag.

The physical computing device may be programmed with executable instructions for (a) receiving information from a storage unit tag on a storage unit for breast milk; (b) storing information from the storage unit tag; and (c) determining the length of storage of a storage unit that includes the storage unit tag. In some embodiments, the physical computing device may be or include a mobile device. In some embodiments, the physical computing device may include a display. In some embodiments, the instructions may include storing and organizing information in a database, wherein the information may come from a plurality of storage unit tags corresponding to a plurality of storage units. In some embodiments, the database may include information from the storage unit tag such as time, date, and/or location of breast pumping and/or time, date, and/or location of storage of the storage unit.

In one aspect, the disclosure provides a method of managing breast milk.

In one aspect, the method of managing breast milk includes a) providing a breast milk management system of the disclosure, wherein the storage unit contains breast milk; b) inputting information from the storage unit tag into the physical computing device; and c) storing the storage unit. The method may further include d) determining a length of storage for the storage unit.

In some embodiments, steps (b)-(d) may be repeated for a plurality of storage units containing breast milk.

In some embodiments, the method may further include calculating the expiration date of milk in the plurality of storage units. In some embodiments, the expiration date may be calculated using storage temperature. For example, if the breast milk is stored at a higher temperature (e.g., within a refrigerator), it may expire more quickly that if it is stored at a lower temperature (e.g., within a freezer). In some embodiments, the method may further include alerting a user to the length of storage for the storage unit. For example, a user may be alerted regarding the expiration date of milk in the storage unit. The user may be alerted prior to the expiration date of the milk in the storage unit.

In some embodiments, the method may further include displaying an inventory of at least a portion of the plurality of storage units.

In some embodiments, the method may further include identifying and/or removing a storage unit from storage for use or disposal.

The term “about,” as used herein, refers to a value that is within 5% or 10% above or below the value being described.

### BRIEF DESCRIPTION OF THE DRAWINGS

The following detailed description of the embodiments of the disclosure will be better understood when read in conjunction with the appended drawings. For the purpose of illustrating the disclosure, there are shown in the drawing embodiments, which are presently exemplified. It should be understood, however, that the disclosure is not limited to the precise arrangement and instrumentalities of the embodiments shown in the drawings.

FIG. 1 shows a schematic of a breast milk management system, according to an example embodiment.

FIG. 2 shows a schematic of the breast milk management pipeline, according to an example embodiment, wherein milk is collected in a storage unit, tagged with a storage unit tag, stored in the freezer, and removed from the freezer for use. A physical computing device in communication with a computer readable memory, e.g., non-transitory storage memory, store information regarding the storage unit tag.

### DETAILED DESCRIPTION

The present disclosure provides a breast milk management system, components thereof, and methods of use thereof. The present breast milk management systems are particularly advantageous for streamlined collection, storage, tracking, and use of stored breast milk. The system advantageously allows a user to optimize freshness of stored breast milk; to pick milk which matches the circadian rhythm of their baby; provides data on storage and use-rate to allow for better planning; and provide better engagement from both parents or multiple caregivers in the feeding routine. It should be understood that references to a “baby” herein are intended to encompass a recipient of breast milk, e.g., an infant or toddler.

The present disclosure is discussed in the context of breast milk. However, the present management system may be used for any purpose where liquids or other foodstuffs are stored and organized in a non-commercial environment, e.g., a household, especially where it is desirable to track individual storage units over time.

### Breast Milk Management

A breast milk management system (e.g., breast milk management system 100) is disclosed herein that is configured to organize and track information about breast milk stored within storage units (e.g., storage units 116) or containers. In some instances, the timing for feeding breast milk depends on how it is stored. For example, freshly expressed breast milk may be used within 4 hours when stored at room temperature (e.g., about 25 °C or cooler).

Alternatively, freshly expressed breast milk may be refrigerated (e.g., at about 4°C) for up to 4 days or frozen (e.g., at about −18 °C) for up to 1 year (with 6 months being preferred). In addition, previously frozen breast milk may be stored for 1-2 hours at room temperature or for one day in a refrigerator. When caregivers have stored multiple storage units of breast milk in multiple locations (e.g., refrigerator and freezer), it can be challenging to organize and use (or dispose of) stored breast milk in a timely manner.

The present disclosure provides a breast milk management system (e.g., breast milk management system 100). The breast milk management system (e.g., breast milk management system 100) is advantageous in that it may receive, store, and retrieve information about pumped breast milk. The disclosed breast milk management system (e.g., breast milk management system 100) is further advantageous in that it can be used with any breast cup and breast pump system.

The breast milk management system (e.g., breast milk management system 100) may include: (a) a storage unit tag (e.g., storage unit tag 122) or label, which may be integral with a storage unit (e.g., storage unit 116) for collecting expressed breast milk or attachable thereto; and (b) a physical computing device (e.g., milk management server 102 and/or user device 108) in communication with a computer readable memory, e.g., a non-transitory storage memory. The physical computing device may include one or more processors in communication with the memory. The memory may be programmed with executable instructions for: (i) receiving information from the storage unit tag and/or pumping session; (ii) storing information from the storage unit tag and/or pumping session; and (iii) retrieving information from the storage unit tag and/or pumping session, e.g., by determining the length of storage of the storage unit.

In some embodiments, the breast milk management system (e.g., breast milk management system 100) may include a plurality of storage units and/or storage unit tags. In some embodiments, the computing device may receive information from a plurality of storage unit tags. In some embodiments, the storage unit tag may include one or more computer-readable and/or human-readable signals. In some embodiments, the storage unit tag may further include computer-readable memory. In some embodiments, the storage unit tag may be programmed to produce an alarm upon receiving a signal.

In some embodiments, the executable instructions may further store information associated with the storage unit tag in a database (e.g., database 114). Information associated with a storage unit tag may include a variety of metrics including the date of milk collection, time of milk collection, location of milk collection, temperature of the location of milk collection, time of storage, date of storage, location of storage, storage temperature, volume, and/or any other relevant metric. Information may be obtained in an automated fashion or by manual entry or by a combination thereof. In some embodiments, the executable instructions may reorganize the information, e.g., in a database, based on the preference of a user.

In some embodiments, the executable instructions may include a variety of calculations. For example, the executable instructions may determine the expiration date of milk stored in a storage unit based on stored information, e.g., date and storage temperature. In some embodiments, the executable instructions may include a programmed alarm to alert the user to impending expiration or expiration. For example, an alarm may be sent, e.g., via text, email, app notification, or audio or visual signal, to notify a user that a storage unit of breast milk is approaching expiration (e.g., an amount of time until an expected expiration date or time is less than a pre-determined threshold). For example, for freezer storage, an alarm may be sent after 1, 2, 3, 4, 5, 6, 7, 8, 9, 10, 11, or 12 months in storage or 1, 2, or 3 weeks or 1, 2, 3, 4, 5, or 6 days prior to expiration (after 12 months) or best by date (6 months). For refrigerator storage, an alarm may be sent after 1, 2, 3, or 4 days in storage or prior to 3, 6, 9, 12, 15, 18, or 21 hours of expiration. Previously frozen milk may also be tracked in the refrigerator in a similar manner. In some embodiments, the executable instructions may send preprogrammed alerts unrelated to an expiration date. The storage location (e.g., storage device 104) may also include a sensor (e.g., sensors 120) in communication with the system to alert the system. In some embodiments, the executable instructions may be configured to allow a user to set a plurality of preprogrammed alerts.

The system may also provide a display (e.g., via input/output device 134 and/or via a similar input/output device of the storage device 104), e.g., in list or graphical format to show the milk storage units in storage. The display may be configured to organize the milk storage units by age to allow for use of the oldest milk first or disposal of milk beyond its expiration date.

In some embodiments, the breast milk management system (e.g., breast milk management system 100) may further be in communication with a breast pump system (e.g., breast pump system 106). In some embodiments, the breast milk management system (e.g., breast milk management system 100) receives information from the breast pump system. This information may include data concerning a pumping session, including the date, time, and duration. The breast pump system may further include one or more sensors (e.g., sensors 130), and information from the one or more sensors may be received by the breast milk management system (e.g., breast milk management system 100). For example, the volume of expressed breast milk in a storage unit may be determined by weight. In some embodiments, information about the weight and/or volume of breast milk in a storage unit may be received by the breast milk management system (e.g., breast milk management system 100). Such information may also be entered manually.

In some embodiments, the breast milk management system (e.g., breast milk management system 100) may further be in communication with a door manager (e.g., a door warden) device (e.g., one of the sensors 120) on the storage location (e.g., storage device 104). The door manager may include a transmission element, e.g., a wireless transmission element, and a receiver element for wirelessly sending signals to and receiving signals from the storage unit tag and/or the computing device. In some embodiments, the door manager may receive and/or transmit information from a storage unit tag based on proximity. In some embodiments, the door manager receives and/or transmits information about the proximity of the storage unit tag, e.g., whether the storage unit tag is in the storage location. In this manner, the door manager can act as a warden or watchdog (e.g., a watchdog sensor) to facilitate tracking and management of stored items.

The system may be employed with ordinary commercial storage locations, e.g., a refrigerator and/or freezer. Alternatively, the storage location (e.g., storage device 104) may be a refrigerator or freezer integrated with the system. Either type of location may include an interior organization system to house milk storage units. For example, the organization system may include labeled slots or compartments for housing one or more milk storage units. When a milk storage unit (e.g., storage unit 116) is placed in the organization system, the storage unit tag (e.g., the storage unit tag 122) may be associated with the slot or compartment of storage (either manually or automatically) to allow for ease in retrieval later. Alternatively, the milk storage units may be stored in the shelves or drawers of an ordinary refrigerator or freezer.

The system may be employed to track any number of storage units, e.g., from 1 to 100 storage units (e.g., from 1 to 2, from 1 to 3, from 1 to 4, from 1 to 5, from 1 to 6, from 1 to 7, from 1 to 8, from 1 to 9, from 1 to 10, from 1 to 11, from 1 to 12, from 1 to 13, from 1 to 14, from 1 to 15, from 1to 16 , from 1 to 17, from 1 to 18, from 1 to 19, from 1 to 20, from 1 to 21, from 1 to 22, from 1 to 23, from 1 to 24, from 1 to 25, from 1 to 26, from 1 to 27, from 1 to 28, from 1 to 29, from 1 to 30, from 1 to 31, from 1 to 32, from 1 to 33, from 1 to 34, from 1 to 35, from 1 to 36, from 1 to 37, from 1 to 38, from 1 to 39, from 1 to 40, from 1 to 41, from 1 to 42, from 1 to 43, from 1 to 44, from 1 to 45, from 1 to 46, from 1 to 47, from 1 to 48, from 1 to 49, from 1 to 50, from 1 to 51, from 1 to 52, from 1 to 53, from 1 to 54, from 1 to 55, from 1 to 56, from 1 to 57, from 1 to 58, from 1 to 59, from 1 to 60, from 1 to 61, from 1 to 62, from 1 to 63, from 1 to 64, from 1 to 65, from 1 to 66, from 1 to 67, from 1 to 68, from 1 to 69, from 1 to 70, from 1 to 71, from 1 to 72, from 1 to 73, from 1 to 74, from 1 to 75, from 1 to 76, from 1 to 77, from 1 to 78, from 1 to 79, from 1 to 80, from 1 to 81, from 1 to 82, from 1 to 83, from 1 to 84, from 1 to 85, from 1 to 86, from 1 to 87, from 1 to 88, from 1 to 89, from 1 to 90, from 1 to 91, from 1 to 92, from 1 to 93, from 1 to 94, from 1 to 95, from 1 to 96, from 1 to 97, from 1 to 98, from 1 to 99, 1, 2, 3, 4, 5, 6, 7, 8, 9, 10, 11 , 12,13 , 14, 15 , 16, 17,18,19 , 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

### Storage Unit Tag

The breast milk management system (e.g., breast milk management system 100) may include a storage unit tag (e.g., storage unit tags 122) or label that can be used to identify a storage unit (e.g., storage units 116) for milk. When a plurality of storage unit tags is employed, each storage unit tag will be unique, or a combination of storage unit tags may be used to produce a unique identifier for each storage unit. Storage unit tags may include any machine or human identifiable structure, e.g., 1D barcode labels, 2D barcode labels (e.g., QR code), radio frequency identifier (RFID) labels, beacon-based labels, near field communication (NFC) tags, Bluetooth® labels (e.g., BLE), magnetic labels, optical character recognition labels, digital labels, character-based labels, color labels, or symbol or graphical labels. It will be understood that the storage unit tag acts as a surrogate for the storage unit and optionally the pumping session within the breast milk management system (e.g., breast milk management system 100). Thus, information from the storage unit tag encompasses not only information about the identity and location of the storage unit tag but also any information in the system regarding the associated storage unit and/or pumping session producing the milk in the storage unit. In some embodiments, the storage unit tag includes a transmission element. In some embodiments, the transmission element is a wireless transmission element. In some embodiments, the transmission element is not a wireless transmission element.

In some embodiments, the breast milk management system (e.g., breast milk management system 100) may include two or more storage unit tags. In some embodiments, the two or more storage unit tags are the same type of storage unit tag, e.g., a first RFID and a second RFID. In some embodiments, the two or more storage unit tags are different types of storage unit tags, e.g., a first RFID and a second character-based label. In some embodiments, one or more storage unit tags are computer readable. In some embodiments, one or more storage unit tags are human-readable. In some embodiments, one storage unit tag is computer-readable, and one storage unit tag is human-readable. A single storage unit tag may also include separate computer and human readable portions.

In some embodiments, the storage unit tag may be removable. In some embodiments, the storage unit tag may be non-removable. In some embodiments, the storage unit tag may be affixed to the outside of the storage unit prior to, during, and/or after a breast pumping session. In some embodiments, the storage unit tag may be integrated into the storage unit. In some embodiments, the storage unit may include one or more storage unit tags. In some embodiments, the storage unit may include two or more storage unit tags. In some embodiments, the storage unit may further include two storage unit tags that are different types of storage unit tags, e.g., a first RFID and a second symbol storage unit tag.

In some embodiments, the storage unit tag may have a moveable component. In some embodiments, the storage unit tag may have a plurality of moveable components. A moveable component may include an overlay that may move slidably along the outer surface of the storage unit tag such sliding the moveable component into a first position may reveal a first storage unit tag (e.g., a first color label), and sliding the moveable component into a second position may reveal a second storage unit tag, (e.g., a second color label). The moveable component may move laterally and/or radially. The movable component may be manually moveable, and/or movable via preprogrammed executable instructions. The storage unit tag may include a physically adjustable tag which has a series of colors or symbols (or combinations of colors, symbols or other visual indicia) so that milk can be visually categorized. For example, colors can be assigned to a particular time of day of pumping or date of pumping, e.g., the month of pumping, to make it easier to find a specific batch or unit of milk. Other visual indicia to differentiate among particular batches or units of milk may be utilized.

In some embodiments, the storage unit tag includes or is connected to a control unit that may be configured to send signals to and receive signals from a physical computing device, e.g., a mobile device. The control unit may include a transmission element, e.g., a wireless transmission element, and a receiver element for wirelessly sending signals to and receiving signals from the computing device.

In some embodiments, the storage unit tag includes or is connected to a power source. In some embodiments, the power source is a battery. In some embodiments, the battery is a single use battery. In some embodiments, the battery is rechargeable.

In some embodiments, the storage unit tag may include or be connected to one or more sensors (e.g., sensors 124). In some embodiments, the sensor is a temperature sensor.

In some embodiments, the storage unit tag may include or be connected to computer readable memory. In some embodiments, the storage unit tag is in communication with a non-transitory storage memory. In some embodiments, the storage unit tag includes a non-transitory storage memory. In some embodiments, the computer readable memory may be configured to save information from a sensor included in or connected to the storage unit tag. In some embodiments, the storage unit tag may be configured to transmit information stored in the digital storage to a door manager and/or physical computing device, e.g., via a mobile device.

In some embodiments, the storage unit tag may further be configured to provide an alarm. In some embodiments, the storage unit tag may be configured to emit an auditory, vibratory, or visual signal.

In some embodiments, the storage unit tag further includes a fastener, such that the storage unit tag may be releasably attached to the storage unit. The fastener may include a clip, a strap, a tie a hook, a zipper, a magnet, a pin, a button, a snap, a hook and loop fastener (e.g., VELCRO®), or a combination thereof. Storage unit tags may also be permanently affixed to a storage unit, e.g., by adhesive or being sealed in a compartment in the storage unit.

### Storage Unit

The breast milk management system (e.g., breast milk management system 100) may include a storage unit (e.g., storage unit 116) or container. The storage unit is a collection container for the milk, such as a bottle or bag. The storage unit may be flexible. The storage unit may be polymeric or fabric. The storage unit may be configured such that any part of the storage unit that contacts the stored milk is a food safe material. The storage unit may not contain BPA. The storage unit may be releasably connected to a breast pump system (e.g., breast pump system 106) including a breast pump and/or breast cup. The storage unit may be releasably connected to a fluid conduit, such as a tube. The storage unit may be configured for ease of cleaning. The storage unit may be reusable. The storage unit may be disposable.

Breast milk may be pumped directly into the storage unit or be transferred to the storage unit from another storage unit.

In some embodiments, the storage unit includes a volume from about 50 mL to about 1500 mL (e.g., about 50 mL to about 100 mL, about 50 mL to about 150 mL, about 50 mL to about 200 mL, about 50 mL to about 250 mL, about 50 mL to about 300 mL, about 50 mL to about 400 mL, about 50 mL to about 500 mL, about 50 mL to about 600 mL, about 50 mL to about 700 mL, about 50 mL to about 700 mL, about 50 mL to about 750 mL, about 50 mL to about 800 mL, about 50 mL to about 900 mL, about 50 mL to about 1000 mL, about 50 mL to about 1100 mL, about 50 mL to about 1200 mL, about 50 mL to about 1300 mL, about 50 mL to about 1400 mL, about 100 mL to about 250 mL, about 100 mL to about 500 mL, about 100 mL to about 750 mL, about 100 mL to about 1000 mL, about 100 mL to about 1250 mL, about 250 mL to about 500 mL, about 250 mL to about 750 mL, about 250 mL to about 1000 mL, about 250 mL to about 1250 mL, about 250 mL to about 1500 mL, about 500 mL to about 750 mL, about 500 mL to about 1000 mL, about 500 mL to about 1250 mL, about 500 mL to about 1500 mL, about 750 mL to about 1000 mL, about 750 mL to about 1250 mL, about 750 mL to about 1500 mL, about 1000 mL to about 1250 mL, about 1250 mL to about 1500 mL, about 50 mL, about 100 mL to about 150 mL, about 200 mL, about 250 mL, about 300 mL, about 350 mL, about 400 mL, about 450 mL, about 500 mL, about 55 mL, about 600 mL, about 650 mL, about 700 mL, about 750 mL, about 800 mL, about 850 mL, about 900 mL, about 950 mL, about 1000 mL, about 1050 mL, about 1100 mL, about 1150 mL, about 1200 mL, about 1250 mL, about 1300 mL, about 1350 mL, about 1400 mL, about 1450 mL, or about 1500 mL).

In some embodiments, the breast milk management system (e.g., breast milk management system 100) includes a plurality of storage units. In some embodiments, breast milk management system (e.g., breast milk management system 100) includes from 1 to 1,000 storage units (e.g., from 1 to 2, from 1 to 3, from 1 to 4, from 1 to 5, from 1 to 6, from 1 to 7, from 1 to 8, from 1 to 9, from 1 to 10, from 1 to 11, from 1 to 12, from 1 to 13, from 1 to 14, from 1 to 15, from 1to 16 , from 1 to 17, from 1 to 18, from 1 to 19, from 1 to 20, from 1 to 21, from 1 to 22, from 1 to 23, from 1 to 24, from 1 to 25, from 1 to 26, from 1 to 27, from 1 to 28, from 1 to 29, from 1 to 30, from 1 to 31, from 1 to 32, from 1 to 33, from 1 to 34, from 1 to 35, from 1 to 36, from 1 to 37, from 1 to 38, from 1 to 39, from 1 to 40, from 1 to 41, from 1 to 42, from 1 to 43, from 1 to 44, from 1 to 45, from 1 to 46, from 1 to 47, from 1 to 48, from 1 to 49, from 1 to 50, from 1 to 51, from 1 to 52, from 1 to 53, from 1 to 54, from 1 to 55, from 1 to 56, from 1 to 57, from 1 to 58, from 1 to 59, from 1 to 60, from 1 to 61, from 1 to 62, from 1 to 63, from 1 to 64, from 1 to 65, from 1 to 66, from 1 to 67, from 1 to 68, from 1 to 69, from 1 to 70, from 1 to 71, from 1 to 72, from 1 to 73, from 1 to 74, from 1 to 75, from 1 to 76, from 1 to 77, from 1 to 78, from 1 to 79, from 1 to 80, from 1 to 81, from 1 to 82, from 1 to 83, from 1 to 84, from 1 to 85, from 1 to 86, from 1 to 87, from 1 to 88, from 1 to 89, from 1 to 90, from 1 to 91, from 1 to 92, from 1 to 93, from 1 to 94, from 1 to 95, from 1 to 96, from 1 to 97, from 1 to 98, from 1 to 99, from 1 to 100, from 1 to 110, from 1 to 120, from 1 to 130, from 1 to 140, from 1 to 150, from 1 to 160, from 1 to 170, from 1to 180 , from 1 to 190, from 1 to 200, from 1 to 220, from 1 to 240, from 1 to 260, from 1 to 280, from 1 to 300, from 1 to 350, from 1 to 400, from 1 to 450, from 1 to 500, from 1 to 550, from 1 to 600, from 1 to 650, from 1 to 700, from 1 to 750, from 1 to 800, from 1 to 850, from 1 to 900, from 1 to 950, from 1 to 1,000, 1, 2, 3, 4, 5, 6, 7, 8, 9, 10, 11, 12 , 13 , 14,15 , 16 , 17, 18,19 , 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000).

In some embodiments, the storage unit is configured to be used directly with a breast cup and/or a breast pump. In some embodiments, the storage unit used to collect breast milk may be storage unit used to store the collected milk in a refrigerator and/or freezer. In some embodiments, the storage unit used to store the collected milk in a refrigerator and/or freezer is not used to collect breast milk directly from a human breast.

In some embodiments, the storage unit is largely rectangular in shape, such that it may easily be stacked with other storage units. In some embodiments, a plurality of storage units may be stacked together in a storage device, e.g., a freezer. In some embodiments, the storage unit is configured such that it takes up a minimal amount of space.

In some embodiments, each storage unit includes a lid, wherein lid includes the pressure outlet, the inlet of the storage unit, and/or the valve of the storage unit. The lid can be integral to or attached to the storage unit. In some embodiments, the lid is releasably attached to the storage unit. In some embodiments, the lid is screwed or snapped onto the storage unit. In particular, the storage unit may include external threads, the lid may include internal threads, and the internal threads and the external threads may provide releasable attachment of the storage unit and lid. In some embodiments, the lid is attached with a magnet, e.g., an electromagnet. In some embodiments, the lid is configured to be opened and/or closed with the electromagnet. In some embodiment, the lid is configured to be opened and/or closed with pressure, e.g., air pressure.

The lid of the storage unit can have a substantially flat or rounded top surface. In some embodiments, the lid of the storage unit may be no wider than the body of the storage unit.

In some embodiments, the storage unit includes a resealable opening. In some embodiments, the resealable opening is liquid-tight, e.g., leak proof. In some embodiments, the opening is sealable with a zippered or snap fit locking mechanism.

In some embodiments, the storage unit, e.g., the lid of the storage unit, includes a pressure outlet. The pressure outlet may or may not include a valve. In some embodiments, the pressure outlet is a breather tube. In some embodiments, the pressure outlet is an orifice, e.g., the storage unit or the lid of the storage unit may include an orifice. A pressure outlet is advantageous in allowing excess pressure to vent as the storage unit is being filled with milk. In some embodiments, the pressure outlet is solely open as the storage unit is being filled, and the pressure outlet is otherwise nominally closed.

In some embodiments, the storage unit may have an integrated storage unit tag. In some embodiments, the storage unit may have a releasably attached storage unit tag. In some embodiments the storage unit may contain two or more storage unit tags that may include two or more computer-readable and/or human-readable signals. In some embodiments, the storage unit may include a storage unit tag that includes one computer-readable signal and one human-readable signal.

In some embodiments, the storage unit may include a location wherein a removable storage unit tag may be releasably secured to the storage unit. In some embodiments, the storage unit may further include a reinforced material at a location where a storage unit tag may be releasably secured to the storage unit.

### Physical Computing Device

The breast milk management system (e.g., breast milk management system 100) includes a physical computing device (e.g., milk management server 102 and/or user device 108), such as a mobile device. The computing device includes processing circuit (e.g., 130) including a processor and a computer readable memory, e.g., capable of executing instructions, receiving and storing data, and analyzing and transmitting information. For example, a physical computing device may include one or more processors, a network communication interface, and one or more computer readable memories to store data and instructions for carrying out the methods of the disclosure.

The physical computing device may be implemented in any suitable manner. For example, it may reside on a single server or computer or be distributed across multiple computers or servers, e.g., in a cloud architecture. The milk management functionalities described herein may be accessed by a standard desktop or terminal by a dedicated program or webpage. The milk management functionalities described herein may also be accessed via a mobile application. Programming may be via software, hardware, or a combination thereof. The network communication interface may allow communication between the physical computing device and the user or between multiple users. The interface may also allow communication between network components of the physical computing device. The network communication interface may also be implemented as several different components, e.g., one for communication with the user and one for communication with processors for executing instructions and/or storage functions. Thus, a mobile device may be used to access the system for data entry (e.g., manual entry of storage unit tag information, date, or milk storage location), detection of storage unit tags, and display of information, e.g., received from cloud storage. The entire program may also be locally stored and executed on a mobile device. Such a mobile device may include a detector, e.g., a camera, RF detector, or other detector for the storage unit tag used.

Any standard network communication protocol may be employed, e.g., Transmission Control Protocol (TCP), Internet Protocol (IP), Global System for Mobile Communications (GSM) based cellular network, Wi-Fi, Bluetooth, and Near Field Communication (NFC). Connections may also be wired, wireless, or a combination thereof.

Any suitable computer readable memory, e.g., non-transitory computer readable memory, may be employed in the physical computing device. Such memories include magnetic disks, optical disks, organic memory, unique alpha numeric IDs, 2-D bar codes, e.g., QR codes, and any other volatile (e.g., Random Access Memory (RAM), flash, and EEPROM) or non-volatile (e.g., Read-Only Memory (ROM)) mass storage system readable by the one or more processors. The memory includes standalone or cooperating or interconnected memories, which may be distributed among multiple interconnected computers or servers that may be local or remote. In one embodiment, the data are stored and/or transmitted with one or more encryption and/or security methods.

A system of the disclosure may also include any other components necessary for operation, e.g., displays, switches, and routers.

In some embodiments, the computing device (e.g., user device 108) may include a processor and instructions, which when executed, cause the processor to track information about a pumping session, including dates, times, volume of milk pumped, the storage unit pumped into, the unique storage unit tag of the storage unit, temperature, time of storage, date of storage, location of storage, and any other information collected, e.g., by a one or more sensors.

In some embodiments, the computing device (e.g., user device 108) may include a processor and instructions, which when executed, cause the processor to track information related to a storage unit tag, including the identity of the storage unit tag, location, storage temperature, and elapsed time of storage. In some embodiments, the processor may use this and other collected information to identify a storage unit of milk to use for feeding a child at a particular time. In some embodiments, the processor analyzes elapsed time and/or collection time of the milk to determine which milk should be utilized.

The system may identify a plurality of storage units at similar points in storage lifetime to allow a user to select from among them or to determine inventory, e.g., the need to store more milk or dispose of expired milk. In some embodiments, the computing device may include instructions that when executed notify a user about the age of one or more milk storage units. The instructions may also allow the user to search for storage units prepared within a date range or stored for a specified date range.

The system will also include a data entry component (e.g., I/O devices 134), e.g., sensor, keyboard, touchscreen, camera, etc.

In some embodiments, the computing device (e.g., user device 108) may include a processor and instructions, which when executed, cause the processor to calculate or store volume of milk extracted and track expression efficiency and monitor it over time. In some embodiments, the calculation of milk extracted and expression efficiency are based on signals received from a breast pump.

In some embodiments, the computing device (e.g., user device 108) may include a processor and instructions, which when executed, transmit a signal to the storage unit tag, whereupon the storage unit tag emits an auditory, vibratory, or visual signal.

In some embodiments, the computing device (e.g., user device 108) may have a share function, such that the data collected by the device and subsequent analysis may be shared with a third party. In some embodiments, the third party may be a caretaker, another parent, a family member, and/or a healthcare provider. Thus, the system may be used to communicate a milk storage unit for feeding an infant when a parent or other primary caregiver is absent.

### Door Manager

The breast milk management system (e.g., breast milk management system 100) may include a door manager device (e.g., one of the sensors 124) that is in communication, e.g., to send and receive signals, with a storage unit tag and/or a physical computing device (e.g., user device 108), e.g., a mobile device. The door manager may include a transmission element, e.g., a wireless transmission element, and a receiver element for wirelessly sending and receiving signals from the storage unit tag and/or the computing device. In some embodiments, the door manager may be further configured to receive wireless signals from a plurality of storage unit tags, and wirelessly transmit those signals to the computing device.

In some embodiments, the door manager may include a detector, e.g., a camera, RF detector, or other detector for the storage unit tag used. The door manager may also monitor whether the storage location is secure, e.g., closed, and within the appropriate temperature range.

In some embodiments, the door manager may be disposed at the threshold of a storage location (e.g., storage device 104), e.g., a refrigerator or freezer. In some embodiments, the door manager is configured such that it may receive a signal from a storage unit tag as it is stored in the storage location. In some embodiments, the door manager may receive a signal from a storage unit tag located in the storage location when the storage location is opened. In some embodiments, the door manager may be located in proximity to an appliance which includes a first storage location and a second storage location, e.g., a single appliance with a refrigerator and a freezer. In some embodiments, the door manager may be able to monitor any storage unit tags located in the first storage location and/or the second storage location.

In some embodiments, the door manager may receive and/or transmit information from a storage unit tag based on proximity. For example, the door manager may receive information from a storage unit tag located within 20 feet, (e.g., within 19 ft, 18 ft, 17 ft, 16 ft, 15 ft, 14 ft, 13 ft, 12 ft, 11 ft, 10 ft, 9 ft, 8 ft, 7 ft, 6 ft, 5 ft, 4 ft, 3 ft, 2 ft, 1 ft, 11 inches, 10 inches, 9 inches, 8 inches, 7 inches, 6 inches, 5 inches, 4 inches, 3 inches, 2 inches, 1 inch, and/or directly next to the door manager device). In some embodiments, the door manager may not receive and/or transmit information from the storage unit tag based on proximity. In some embodiments, the door manager may receive and/or transmit information about the proximity of a storage unit tag.

### Methods of Use

The present disclosure provides methods of storing, organizing, and managing breast milk or a household foodstuff. The present disclosure provides a method of breast milk management by: providing a breast milk management system and storage unit or container containing human breast milk and having a storage unit tag; (b) the physical computing device receiving information from the storage unit tag; and (c) storing the storage unit in a storage location. The physical computing device may also receive information about the pumping session producing the milk in the storage unit.

In some embodiments, the physical computing device further (stores information about the storage unit tag and retrieves information about the storage unit tag. The method may also include transmitting information to a user about the age of one or more milk storage unit in storage.

In some embodiments, the method is repeated for a plurality of storage units and/or storage unit tags.

In some embodiments, the method includes organizing and storing information received from the storage unit tag into a database. In some embodiments, the method includes reorganizing the information in the database dependent on the preferences of a user. In some embodiments, the method includes organizing information from a plurality of storage unit tags in a database. In some embodiments, the method may include updating stored information from a storage unit tag with new information from the storage unit tag, e.g., change in storage location, such as from freezer to refrigerator or from storage location to use or disposal. In some embodiments, this new information may be received from the storage unit tag, from a manual entry, and/or from one or more additional processors in communication with the breast milk management system (e.g., breast milk management system 100). The method may include organizing the database such that the oldest stored storage unit or a storage unit from a selected time period may be the storage unit identified for use, e.g., first in first out. The database may be organized based on the time of day that milk was expressed, e.g., morning, midday, or evening. The method may include organizing the database to account for a plurality of factors, e.g., first in first out and time of day.

In some embodiments, the method includes the system sending an alarm or a plurality of alarms. In some embodiments, the plurality of alarms may be tailored to the preference of a user. In some embodiments, the storage unit tag may produce an alarm upon receiving a signal from the system.

In some embodiments, the information received from the storage unit tag is received by the computing device via network] communication, e.g., from a mobile device. In some embodiments, the information received from the storage unit tag received by the computing device is manually entered. In some embodiments, the computing device receives information from the storage unit tag via network and/or manual communication. In some embodiments, the physical computing device may include a detector, e.g., a camera, RF detector, or other detector for the storage unit tag used. In these embodiments, the method may include receiving information about the storage unit tag via the detector.

In some embodiments, the breast milk management system (e.g., breast milk management system 100) is in communication with a breast pump. In this embodiment, the method may include receiving, storing, and retrieving information from the breast pump, wherein the information may be associated with a storage unit tag and/or storage unit. For example, the information received from the breast pump may be associated with the storage unit tag that storage unit tags the storage unit wherein the milk from the breast pumping session is stored.

In some embodiments, the breast milk management system (e.g., breast milk management system 100) is in communication with a door manager. In this embodiment, the method may include receiving, storing, and retrieving information from a storage unit tag from the door manager. In some embodiments, the breast milk management system (e.g., breast milk management system 100) may receive information from a plurality of storage unit tags from the door manager. In some embodiments, the door manager may provide the breast milk management system (e.g., breast milk management system 100) with updates as to a change in the storage status of the storage unit tag. In some embodiments, the executable instructions may recalculate any calculations and/or reorganize the database based on information received from the door manager.

In some embodiments, the method may be performed outside of a neonatal intensive care unit. In some embodiments, the method may be performed outside of a hospital. In some embodiments, the method may be performed in a residential setting.

In some embodiments, the method may further include the breast milk management system (e.g., breast milk management system 100) transmitting information about a storage unit tag. In some embodiments, information about the storage unit tag may be transmitted to a healthcare provider, a family member, and/or a caregiver.

The method may further display information about a storage unit associated with a storage unit tag to a user, e.g., information on pumping (time, date, location, volume, etc.) or storage of the storage unit (time, date, location, etc.). The method may allow the user to display information about a plurality of storage units associated with storage unit tags to a user, e.g., to allow a user to review and select a particular storage unit for use or disposal. The method may also automatically identify or suggest a storage unit for use, e.g., based on first in, first out.

The method may be adjusted to replace the breast milk with any household foodstuff.

### Kits

In another aspect, the disclosure provides kits. Kits may include two or more unconnected components of a breast milk management system (e.g., breast milk management system 100). A kit may include any two or more, e.g., all, of a storage unit, a storage unit tag, a physical computing device, and a door manager.

### Example Breast Milk Management System

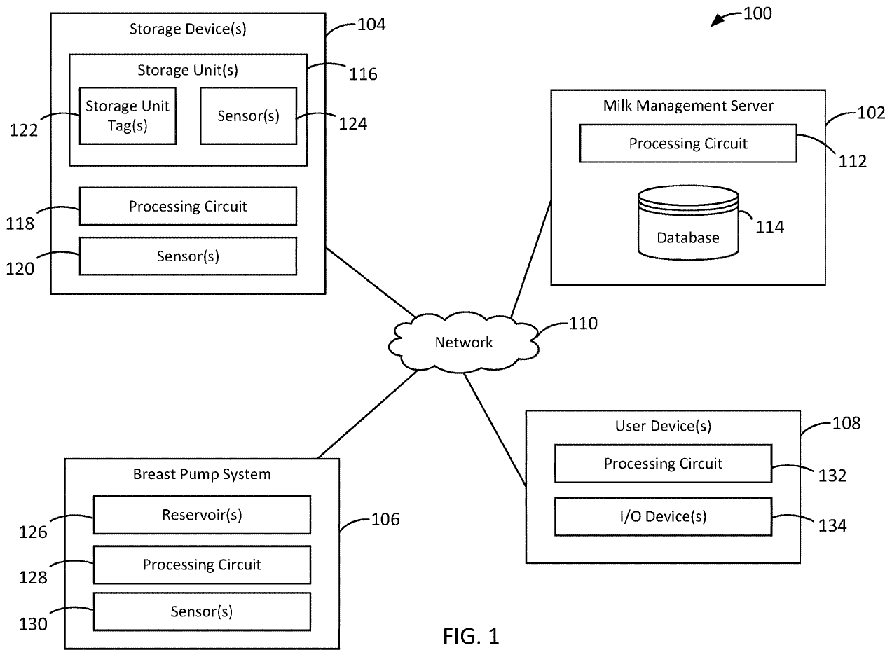

FIG. 1 shows a breast milk management system 100 providing the various components and functionalities described above, according to an example embodiment. The breast milk management system 100 includes a milk management server 102, one or more storage devices 104, a breast pump system 106, and one or more user devices 108. In some instances, the milk management server 102, the one or more storage devices 104, the breast pump system 106, and/or the one or more user devices 108 are each in communication with one another via a network 110, such as a wireless network (e.g., Wi-Fi, Bluetooth) and/or a wired network. It should be appreciated that, in some instances, the breast pump system 106 may include more or fewer components.

The milk management server 102 includes a processing circuit 112 configured to perform various assessments and functionalities described herein. For example, the processing circuit 112 may include one or more processors and one or more memories storing instructions thereon that, when executed by the one or more processors, cause the one or more processing circuits to perform various functions described herein. In some instances, the processing circuit 112 may further include one of more communication interfaces configured to allow for the processing circuit 112 to communicate with other devices over a communications network (e.g., Bluetooth, Wi-Fi, near-field communication, etc.).

The milk management server 102 further includes a database 114 configured to store a variety of collected information pertaining to stored breast milk within the breast milk management system 100, as described herein. For example, in some instances, the milk management server 102 may receive various information pertaining to stored breast milk, along with a corresponding unique identifier associated with a storage unit tag 122 of a storage unit 116, from any of the storage devices 104, the breast pump system 106, and/or the user devices 108. The milk management server 102 may then store the information pertaining to the stored breast milk correlated to the particular storage unit 116 via the storage unit tag 122 to allow for the information pertaining to the storage unit 116 to be tracked over time.

The storage devices 104 may comprise one or more refrigerators, freezers, and/or other temperature-and/or climate-controlled storage devices configured to store one or more individual storage units 116. In some instances, each storage device 104 may include a processing circuit 118 configured to control various functionalities of the storage device 104 (e.g., a temperature or other climate parameter of a storage cavity within the storage device 104) and/or to transmit various storage-related information to an external system (e.g., the milk management server 102 and/or an application running on the user device 108) via the network 110.

For example, the processing circuit 118 may similarly include one or more processors and one or more memories storing instructions thereon that, when executed by the one or more processors, cause the one or more processing circuits to perform various functions described herein. In some instances, the processing circuit 118 may further include one of more communication interfaces configured to allow for the processing circuit 118 to communicate with other devices over a communications network (e.g., Bluetooth, Wi-Fi, near-field communication, etc.).

Each of the storage devices 104 may additionally include various sensors 120 that are configured to communicate various sensor data captured within the storage device 104 and/or associated with the individual storage units 116 stored within the storage devices 104 to an external system (e.g., the milk management server 102 and/or an application running on the user device 108) via the network 110. For example, in some instances, the one or more sensors 120 are configured to detect, sense, or otherwise measure one or more of a weight, a volume, a temperature, optical characteristics, or any other relevant information regarding milk stored within the storage device 104 and/or the individual storage units 116.

The individual storage units 116 may each comprise a container for storing milk, such as a milk storage bag, a bottle, or another type of reservoir. In some instances, each of the individual storage units 116 includes a corresponding storage unit tag 122. The storage unit tag 122 may be or include a label or unique identifier used to identify and track the corresponding individual storage unit 116, as described above. In some instances, the storage unit tag 122 may additionally be configured to store various information associated with the corresponding storage unit 116 within an internal memory, as also described above.

In some instances, each of the individual storage units 116 may similarly include one or more sensors 124 that are configured to communicate various sensor data captured within or otherwise associated with the corresponding individual storage unit 116 to an external system or device (e.g., the milk management server 102, the corresponding storage device 104, and/or an application running on the user device 108) via a wired or wireless connection (e.g., the network 110). For example, in some instances, the one or more sensors 124 are configured to detect, sense, or otherwise measure one or more of a weight, a volume, a temperature, a pH, a viscosity, optical characteristics, or any other relevant information regarding milk stored within the individual storage units 116.

The breast pump system 106 may include one or more breast pumps, pressure sources, reservoirs, and fluid conduit to perform a breast pumping operation to induce lactation and obtain breast milk from a human subject (a mother, donor, care provider, etc.) to be stored for later use (e.g., via one of the storage units 116) and eventual consumption by a receiving subject (as may also be referred to as a recipient, subject, baby, infant, toddler, etc.). In some instances, the breast pump system 106 is configured to temporarily store the pumped milk within one or more reservoirs 126 (e.g., milk bottles or other storage features).

In some instances, the breast pump system 106 includes a processing circuit 128 configured to control the breast pump system 106 to perform the breast pumping operation and to transmit various information pertaining to the pumped milk to an external system (e.g., the milk management server 102, a user device 108) via the network 110. For example, the processing circuit 128 may similarly include one or more processors and one or more memories storing instructions thereon that, when executed by the one or more processors, cause the one or more processing circuits to perform various functions described herein. In some instances, the processing circuit 128 may further include one of more communication interfaces configured to allow for the processing circuit 128 to communicate with other devices over a communications network (e.g., Bluetooth, Wi-Fi, near-field communication, etc.).

In some instances, the breast pump system 106 similarly includes one or more sensors 130 that are configured to communicate various sensor data captured within the reservoirs 126 and/or otherwise associated with the corresponding breast pump system 106 to an external system or device (e.g., the milk management server 102 and/or an application running on the user device 108) via a wired or wireless connection (e.g., the network 110).

For example, in some instances, the one or more sensors 124 are configured to detect, sense, or otherwise measure one or more of a weight, a volume, a temperature, a pH, a viscosity, optical characteristics, or any other relevant information regarding milk flowing, pumped, and/or stored within the breast pump system 106.

Each of the user devices 108 may be a personal user device, such as a laptop, a mobile phone, a smart device, and/or any other suitable device configured to operate or otherwise access an application provided by and/or for use with the breast milk management system 100 described herein. It should be appreciated that, in some instances, the breast milk management system 100 may include only a single user device 108.

In some instances, each user device 108 similarly includes a processing circuit 132 configured to control various functionality of the user device 108 to access the application to perform various functionalities described herein. For example, the processing circuit 132 may similarly include one or more processors and one or more memories storing instructions thereon that, when executed by the one or more processors, cause the one or more processing circuits to perform various functions described herein. In some instances, the processing circuit 132 may further include one of more communication interfaces configured to allow for the processing circuit 128 to communicate with other devices over a communications network (e.g., Bluetooth, Wi-Fi, near-field communication, etc.).

In some embodiments, each user device 108 may include one or more input/output (I/O) devices 134 configured to allow for user to selectively operate and/or interact with the user device 108 to access the application and perform the various functionalities described herein. For example, the one or more I/O devices 134 may include a display, one or more buttons (e.g., a power button, a volume button), a touchscreen, an audio speaker, an alarm, and/or any other suitable I/O devices.

### Example Breast Milk Management System Process Flow

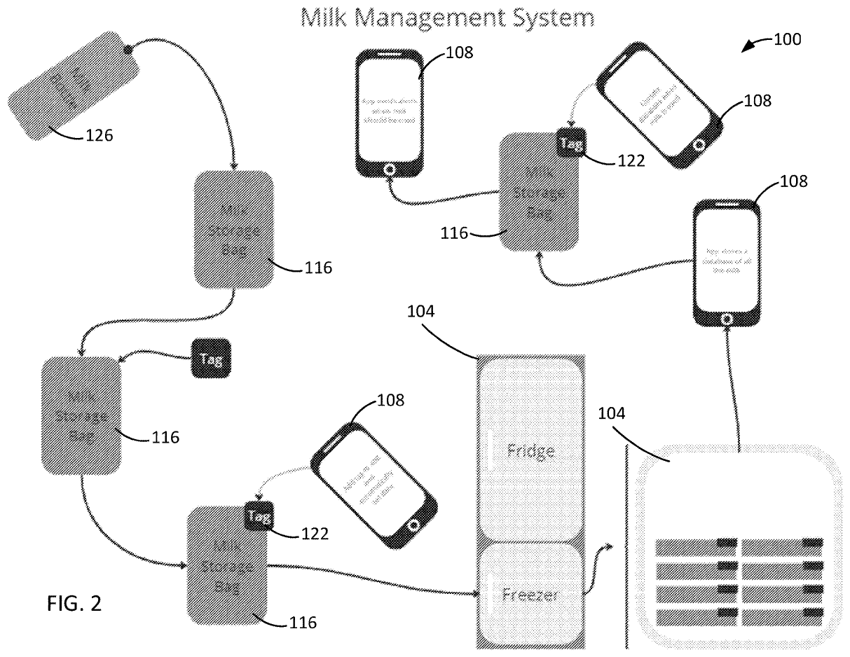

FIG. 2 shows a process flow of managing stored breast milk using the breast milk management system 100, according to an example embodiment. Milk from a reservoir 126 (e.g., a milk bottle) of the breast pump system 106 is transferred into a storage unit 116 (e.g., a milk storage bag). A storage unit tag 122 (e.g., a label or tag) is then attached to the storage unit 116. The storage unit tag 122 is scanned by an application on the user device 108 (e.g., via one of the I/O devices 134) to enter information from the storage unit tag 122 or associated with the corresponding storage unit 116 into the application. The entry may automatically include information about the date and time of scanning, the date and time of the corresponding pumping session in which the breast milk was pumped, and/or various other information associated with the pumping session (e.g., the mother's dietary habits leading up to the pumping session). The storage unit 116 is then be placed in the storage device 104 (e.g., a refrigerator or freezer) storing a plurality of similar storage units 116 until they are retrieved for later use or disposal.

In some instances, the application may store all the relevant information pertaining to the breast milk management system 100 locally (e.g., within a memory of the processing circuit 132 of the user deice 108). In some other instances, the application may automatically transmit the various information to the milk management server 102 to be remotely stored within the database 114.

Accordingly, the breast milk management system 100 has a database (e.g., within the user device 108 and/or the database 114) of all stored milk bags and their corresponding stored information. In some instances, breast milk management system 100 (e.g., the milk management server 102 and/or the application running locally on the user device 108) is configured to send various notifications and alarms to a user based on various gathered information (e.g., to notify a user when a particular bag should or should not be used).

In some instances, once a storage unit 116 is removed from the storage device 104, the storage unit tag 122 (e.g., the label) may again be scanned to remove it from the database.

### Example Breast Milk Management System Use Cases

Now that the example breast milk management system 100 and corresponding process flow have been described above, a variety of beneficial use cases are provided below. It should be appreciated that these are provided as examples and are in no way meant to be limiting.

As a first example, in some instances, the breast milk management system 100 can be utilized to track milk freshness and expected expiration of milk store within the various storage units 116 within the storage devices 104. For example, the pumping date, pumping time, and pumping conditions associated with the milk within the storage units 116 can be logged (e.g., within a memory device of the storage unit tag 122, within the application on the user device, and/or within the database 114), and a user (e.g., a parent) can track (e.g., via the application accessed using the user device 108) when the milk was pumped, the exact duration it has been stored, and whether it is still fresh. Accordingly, by knowing the aforementioned information, the user can prioritize particular storage units 116 for use based on their age (e.g., using the freshest milk first, using the milk that is closest to expiring first) and/or know when milk within certain storage units 116 should be discarded. As such, the breast milk management system 100 beneficially reduces the risk of feeding potentially spoiled milk to the baby and helps with inventory management of stored milk.

In some instances, the breast milk management system 100 can be utilized to coordinate pumping times of stored milk with feeding schedules of infants. For example, because the pumping times can be tracked, as discussed above, the user can utilize the application on the user device 108 to identify which storage unit 116 containing pumped milk should be used for each feeding session based on the time at which the feeding session is to take place and the time of day at which the milk was pumped. That is, the composition of breast milk naturally changes throughout the day to include different nutrients that are more suitable for the feeding baby's needs. For example, the breast milk may include more melatonin when pumped at night versus when the breast milk is pumped in the morning.

Accordingly, by tagging milk with the time of day it was pumped, the breast milk management system 100 can match an appropriate batch of pumped milk to a baby's feeding schedule (e.g., by identifying a storage unit 116 storing milk pumped at the same or a similar time of day). As an example, milk pumped at night can be prioritized or otherwise identified for nighttime feeding on another date. This synchronization aids in optimizing feeding schedules and can allow for the baby to receive milk that may be better suited to their needs at different times of the day.

As another example, the breast milk management system 100 can be utilized to track potential allergens within pumped milk based on correlated diet information associated with the pumping user. For example, in addition to the pumping time and pumping date, the user can provide additional information associated with their diet at the time of pumping via the application on the user device 108, and this information can similarly be stored within the system and correlated to the corresponding storage unit 116 and storage unit tag 122. In some instances, one or more sensors (e.g., sensors 124, sensors 130) may additionally or alternatively be configured to detect potential allergen contamination within the breast pump system 106 and/or within the storage unit 116. Accordingly, if the user consumes potential allergens (e.g., dairy, nuts, etc.), the user may provide this information to the system and/or the system may detect these allergens, and the system may provide an alert to the user (e.g., via the application on the user device 108) to avoid using that milk if the baby has related food sensitivities or allergies, thereby avoiding potential allergic reactions in the baby.

In some instances, the breast milk management system 100 can be utilized to track a baby's response to different pumping sessions with different pumping information. For example, a user may input (e.g., via the application on the user device 108) notes before, during, and/or after feeding sessions using pumped milk tracked via the breast milk management system 100, and the breast milk management system 100 may correlate different characteristics of the pumped milk (e.g., the mother's diet, the pumping time of day, the duration of storage, etc.) with positive and/or negative responses by the baby. The breast milk management system 100 may then provide various information and suggestions pertaining to the baby's preferred milk characteristics to the mother, such that she can track these characteristics and adjust her pumping schedule or settings accordingly. For example, the breast milk management system 100 may detect and provide and alert to the mother indicating that milk from morning sessions appears to be more satisfying for the baby (e.g., morning session milk has generally resulted in better responses from the baby, e.g., higher consumption). These types of alerts aid in allowing mothers to understand their baby's preferences and tailor feeding.

As another example, the breast milk management system 100 allows for improved caregiver coordination. For example, in situations where multiple caregivers are involved in feeding a baby, tagging the stored milk with detailed session information and providing that information back to each user (e.g., via corresponding user devices 108 using the application) ensures that each caregiver is on the same page regarding which milk to use and when to use it. Accordingly, the breast milk management system 100 allows for improved communication and coordination among caregivers, ensuring the baby receives an appropriate unit of milk at an appropriate time (e.g., milk with higher melatonin being provided for a nocturnal feeding session).

Accordingly, the breast milk management system 100 allows for effective and automated storage monitoring and provides a variety of beneficial insights to users. The information from the storage unit tags can be synced with an app or cloud-based system (e.g., the milk management server 102), and mothers can view what milk they have stored, receive alerts, or get a variety of recommendations based on the stored information. As such, the breast milk management system 100 offers convenience and reassurance, including for caregivers on travel or those frequently away from their baby.

As utilized herein with respect to numerical ranges, the terms “approximately,” “about,” “substantially,” and similar terms generally mean ±5% or 10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that can result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

References herein to the positions of elements (e.g., “top,” “bottom,” “above,” “below”) are merely used to describe the orientation of various elements in the FIGURES. It should be noted that the orientation of various elements can differ according to other exemplary embodiments, and that such variations are intended to be encompassed by the present disclosure. Additionally, the terms “connected” or “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

It is important to note that any element disclosed in one embodiment may be incorporated or utilized with any other embodiment disclosed herein. The systems and methods described herein may be embodied in other specific forms without departing from the characteristics thereof. The foregoing implementations are illustrative rather than limiting of the described devices and methods. The scope of the devices and methods described herein is thus indicated by the appended claims, and changes that come within the meaning and range of equivalency of the claims are embraced therein.

What is claimed is:

1. A breast milk management system comprising:
a storage unit for storing breast milk, the storage unit including a storage unit tag; and
at least one processing circuit comprising at least one processor and at least one memory having instructions thereon that, when executed by the at least one processor, cause the at least one processor to:
receive information associated with the breast milk based on the storage unit tag being scanned by a user device of a user;
determine a characteristic of the breast milk based on the information associated with the breast milk; and
provide an indication of the characteristic to the user via the user device.

2. The breast milk management system of claim 1, wherein the characteristic is a duration of storage of the breast milk.

3. The breast milk management system of claim 1, wherein the characteristic of the breast milk is an expected expiration date or time of the breast milk.

4. The breast milk management system of claim 3, wherein the indication is an alert or alarm generated based on an amount of time until the expected expiration date or time being less than a pre-determined threshold.

5. The breast milk management system of claim 1, wherein the characteristic is a time of day at which the breast milk was pumped, and the indication is provided based on the user accessing the storage unit at a corresponding time of day.

6. The breast milk management system of claim 1, wherein the characteristic is an allergen content of the breast milk, and the indication is an alert regarding the allergen content.

7. The breast milk management system of claim 6, wherein the allergen content is determined based on a user-input indication of a diet of a subject during or before pumping.

8. The breast milk management system of claim 1, wherein the indication is a first indication and the instructions, when executed by the at least one processor, further cause the at least one processor to:
receive feeding information associated with one or more feeding sessions of a subject, the feeding information including breast milk characteristics of breast milk fed to the subject and corresponding response information indicative of a response of the subject to the one or more feeding sessions; and
provide a second indication to the user device, the second indication indicating one or more breast milk characteristics associated with positive responses by the subject.

9. The breast milk management system of claim 1, wherein the user device of the user is a first user device of a first user, and the instructions, when executed by the at least one processor, further cause the at least one processor to provide the indication to a second user device of a second user.

10. The breast milk management system of claim 1, further comprising a plurality of storage units for storing breast milk and comprising a plurality of storage unit tags, with each storage unit corresponding to one storage unit tag.

11. The breast milk management system of claim 10, wherein the instructions, when executed by the at least on processor, further cause the at least one processor to store and organize information associated with the plurality of storage unit tags in a database.

12. The breast milk management system of claim 11, wherein the instructions, when executed by the at least on processor, further cause the at least one processor to display an inventory of the plurality of storage units including characteristics of breast milk within each storage unit.

13. The breast milk management system of claim 10, wherein the plurality of storage units are stored within a storage device, the storage device being at least one of a refrigerator or a freezer and comprising a temperature sensor.

14. The breast milk management system of claim 1, wherein the storage unit tag comprises a 1D barcode label, a 2D barcode label, a radio frequency label (RFID), a beacon-based label, a near field communication (NFC) tag, a Bluetooth label, a magnetic label, an optical character recognition label, a digital label, a character-based label, a color label, or a symbol label.

15. The breast milk management system of claim 1, wherein the information associated with the breast milk comprises one or more of a time of breast pumping of the breast milk, a date of breast pumping of the breast milk, a location of breast pumping of the breast milk, a volume of breast milk contained in the storage unit, a time of storage of the storage unit, a date of storage of the storage unit, or a location of storage of the storage unit.

16. The breast milk management system of claim 1, wherein the at least one processing circuit is implemented within one of a breast milk management server or the user device.

17. A method of managing breast milk comprising:
receiving information associated with breast milk stored within a storage unit having a storage unit tag based on the storage unit tag being scanned by a user device of a user;
determining a characteristic of the breast milk based on the information associated with the breast milk; and
providing an indication of the characteristic to the user device.

18. The method of claim 17, wherein the information includes a storage temperature of the breast milk captured by a temperature sensor, and the characteristic is an expiration date calculated using the storage temperature.

19. The method of claim 17, further comprising removing the storage unit from storage for use or disposal.

20. The method of claim 17, further comprising:
receiving feeding information associated with one or more feeding sessions of a subject, the feeding information including breast milk characteristics of breast milk fed to the subject and corresponding response information indicative of a response of the subject to the one or more feeding sessions; and
providing a second indication to the user device, the second indication indicating one or more breast milk characteristics associated with positive responses by the subject.
